(19) United States
(12) Patent Application Publication
(10) Pub. No.: US 2023/0218425 A1
(43) Pub. Date: Jul. 13, 2023

(54) FLUID COLLECTION DEVICES INCLUDING A DRY ADHESIVE REGION, AND RELATED SYSTEMS AND METHODS

(71) Applicant: PUREWICK CORPORATION, Covington, GA (US)

(72) Inventors: Briana Lewis, Forest Park, GA (US); Nathaniel Barnes, Covington, GA (US); Danielle Glickstein, Atlanta, GA (US); Ash-Shakur Abdullah Manning, Snellville, GA (US); Shahab Siddiqui, Lawrenceville, GA (US)

(21) Appl. No.: 18/150,360

(22) Filed: Jan. 5, 2023

Related U.S. Application Data
(60) Provisional application No. 63/299,208, filed on Jan. 13, 2022.

(51) Int. Cl. A61F 5/443; A61F 5/451

(52) U.S. Cl. CPC A61F 5/443; A61F 5/451

(57) ABSTRACT

Example fluid collection devices, and related systems and methods of use are described. The fluid collection device includes a fluid impermeable barrier having a rear region and a front region at least partially defining an opening and positioned on the fluid impermeable barrier to be at least proximate to a urethra of a user. The fluid impermeable barrier at least partially defines a chamber and an aperture sized and dimensioned to receive a conduit therethrough. The fluid collection device includes a fluid permeable body positioned at least partially within the chamber to extend across at least a portion of the opening and configured to wick fluid away from the opening. The fluid collection device includes a dry adhesive region positioned on the rear region of the fluid impermeable barrier at least partially distal to the opening to interface a garment worn by the user.

### CROSS-REFERENCE TO RELATED APPLICATIONS

This application claims priority to U.S. Provisional Patent Application No. 63/299,208 filed on Jan. 13, 2022, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices having a dry adhesive region, and related systems and methods. In an embodiment, a fluid collection device includes a fluid impermeable barrier having a rear region and a front region at least partially defining an opening and positioned on the fluid impermeable barrier to be at least proximate to a urethra of a user. The fluid impermeable barrier further at least partially defines a chamber and an aperture sized and dimensioned to receive a conduit therethrough. The fluid collection device also includes a fluid permeable body positioned at least partially within the chamber to extend across at least a portion of the opening and configured to wick fluid away from the opening. The fluid collection device also includes a dry adhesive region positioned on the rear region of the fluid impermeable barrier at least partially distal to the opening to interface a garment worn by the user over the fluid collection device.

In an embodiment, a method of collecting fluid from a user is described. The method includes positioning an opening of a fluid collection device at least proximate to a urethra of the user. The fluid collection device includes a fluid impermeable barrier defining a chamber, the opening having fluid communication with the chamber, and an aperture having a conduit extending therethrough in fluid communication with the chamber. The method includes positioning a garment on the user over the fluid collection device interfacing a dry adhesive region positioned on a rear region of the fluid impermeable barrier at least partially distal to the opening. The method includes collecting fluid voided or discharged by the user in the chamber of the fluid collection device.

In an embodiment, a method of forming a fluid collection device is described. The method includes forming a fluid impermeable barrier having a rear region and a front region at least partially defining an opening and positioned on the fluid impermeable barrier to be at least proximate to a urethra of a user. The fluid impermeable barrier further at least partially defines a chamber and an aperture sized and dimensioned to receive a conduit therethrough. The method includes positioning a fluid permeable body at least partially within the chamber to extend across at least a portion of the opening and configured to wick fluid away from the opening. The method includes positioning a dry adhesive region on the rear region of the fluid impermeable barrier at least partially distal to the opening to interface a garment worn by the user.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a system for fluid collection, according to an embodiment.

FIG. 2A is a front isometric view of a fluid collection device, according to an embodiment.

FIG. 2B is a rear view of the fluid collection device of FIG. 2A.

FIG. 2C is a cross-sectional view of the fluid collection device of FIG. 2A taken along line 2-2 thereof.

FIG. 2D is an exploded view of the fluid collection device of FIG. 2A.

FIG. 3A is an enlarged view of a portion of a dry adhesive region of a fluid collection device, according to an embodiment.

FIG. 3B is an enlarged view of a portion of a dry adhesive region of a fluid collection device, according to an embodiment.

FIG. 4 is a flow diagram of a method to collect fluid, according to an embodiment.

FIG. 5 is a flow diagram of a method of forming a fluid collection device, according to an embodiment.

FIG. 6A is an isometric top view of a fluid collection device, according to an embodiment.

FIG. 6B is an isometric bottom view of the fluid collection device of FIG. 6A.

FIGS. 7A and 7B are an isometric view and a schematic cross-sectional view, respectively, of a fluid collection device, according to an embodiment.

### DETAILED DESCRIPTION

Embodiments disclosed herein include fluid collection devices having a dry adhesive region, and related systems and methods. Conventional fluid (e.g. urine) collection devices are easily moved out of place during use. Wet adhesives securing the fluid collection devices in place may cause discomfort and/or irritation to the user, and also may damage clothing to which the wet adhesive is secured. Described herein are fluid collection devices having a dry adhesive region on the rear region of the fluid collection device. The dry adhesive region may be positioned to interface with clothing (e.g., undergarment) to prevent or inhibit the fluid collection device from being moved out of place. In at least one, some, or all embodiments, the dry adhesive region on the fluid collection device results in the technical effect of sticking to the inside of the undergarment for improved placement, comfort, and/or securement of the fluid collection device in a preferred position, especially when the user is sleeping. The dry adhesive region may include a biomimetic dry adhesive region. For example, the dry adhesive region may include biomimetic synthetic gecko material such as micro or nanometer-scaled polymer brushes or hairs on the dry adhesive region. In some embodiments, the dry adhesive region may include micro or nanometer-scaled patterning formed by, for example, lithography or lasers. In some embodiments, the fluid collection device includes a flexible spine for more improved securement.

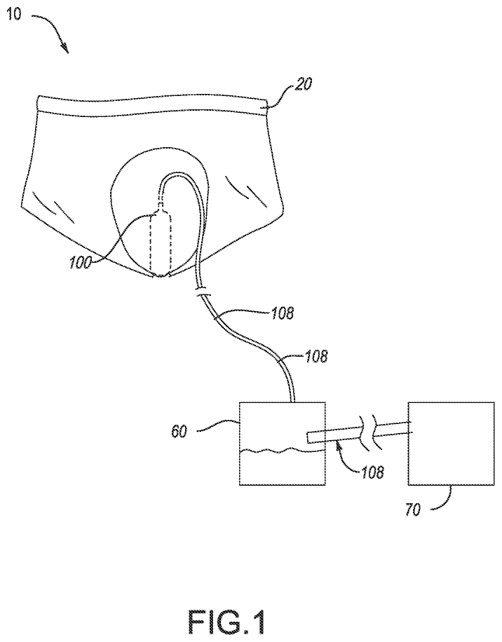

FIG. 1 is a block diagram of a system 10 for fluid collection, according to an embodiment. The system 20 includes a fluid collection device 100, a fluid storage container 60, and a vacuum source 70. The fluid collection device 100, the fluid storage container 60, and the vacuum source 70 may be in fluid communication with each other via one or more conduits 108. For example, fluid collection device 100 may be operably coupled to (e.g., in fluid communication with) one or more of the fluid storage container 60 or the portable source 70 via the conduits 108. A garment 20 (e.g., undergarment) may be worn by a user (not shown) and may interface at least a portion of the fluid collection device 100. Embodiments of garments are disclosed in PCT Patent Application No. PCT/US21/44699, filed on Aug. 5, 2021, the disclosures of which are incorporated herein, in its entirety, by this reference.

Fluid (e.g., urine or other bodily fluids) collected in the fluid collection device 100 may be removed from the fluid collection device 100 via the conduit 108 which protrudes into an interior region of the fluid collection device 100. For example, a first open end of the conduit 108 may extend into the fluid collection device 100 to a reservoir therein. The second open end of the conduit 108 may extend into the fluid collection device 100 or the vacuum source 70. The suction force may be introduced into the interior region of the fluid collection device 100 via the first open end of the conduit 108 responsive to a suction (e.g., vacuum) force applied at the second end of the conduit 108. The suction force may be applied to the second open end of the conduit 108 by the vacuum source 70 either directly or indirectly.

The suction force may be applied indirectly via the fluid storage container 60. For example, the second open end of the conduit 108 may be disposed within the fluid storage container 60 and an additional conduit 108 may extend from the fluid storage container 60 to the vacuum source 70. Accordingly, the vacuum source 70 may apply suction to the fluid collection device 100 via the fluid storage container 60. In some examples, the suction force may be applied directly via the vacuum source 70. For example, the first open end of the conduit 108 may be disposed in the fluid collection device 100 and the second open end of the conduit 108 may be disposed within the vacuum source 70. An additional conduit 108 may extend from the vacuum source 70 to a point outside of the fluid collection device 100, such as to the fluid storage container 60. In such examples, the vacuum source 70 may be disposed between the fluid collection device 100 and the fluid storage container 60.

In examples, the fluid storage container 60 may include a bag (e.g., drainage bag), a bottle or cup (e.g., collection jar), tubing, or any other container for storing bodily fluids such as urine. In examples, the conduit 108 may extend from the fluid collection device 100 and attach to the fluid storage container 60 at a first point therein. An additional conduit 108 may attach to the fluid storage container 60 at a second point thereon and may extend and attach to the vacuum source 70. For example, the fluid storage container 60 may include a container in fluid communication with a first conduit section that is also in fluid communication with the fluid collection device 100. The container may be in fluid communication with a second section of the conduit 108 that is also in fluid communication with the vacuum source. In such examples, the vacuum source 70 may provide a vacuum/suction through the container to the fluid collection device 100 to provide suction in the chamber of the fluid collection device. Accordingly, a vacuum (e.g., suction) may be drawn through fluid collection device 100 via the fluid storage container 60. As the fluid is drained from the chamber, the fluid may travel through the first section of conduit to the fluid storage container where it may be retained. Fluid, such as urine, may be drained from the fluid collection device 100 using the vacuum source 70.

The vacuum source 70 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 70 may provide a vacuum or suction to remove fluid from the fluid collection device 100. In examples, the vacuum source 70 may be powered by one or more of a power cord (e.g., connected to a power socket), one or more batteries, or even manual power (e.g., a hand operated vacuum pump). The vacuum sources 70 disclosed herein may include one or more of a switch, a button, a plug, a remote, or any other device suitable to activate the vacuum source 70.

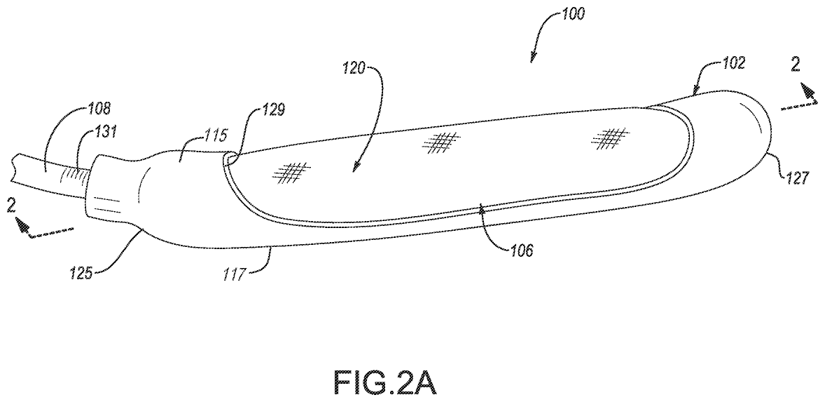

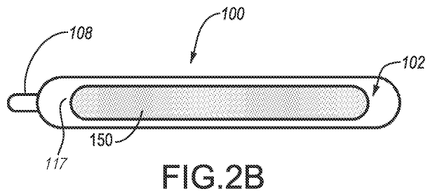

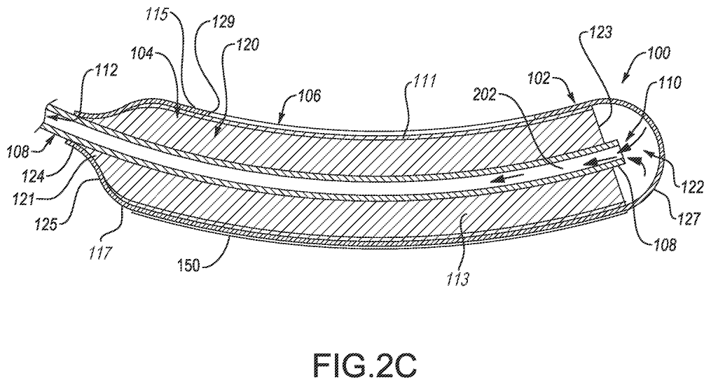

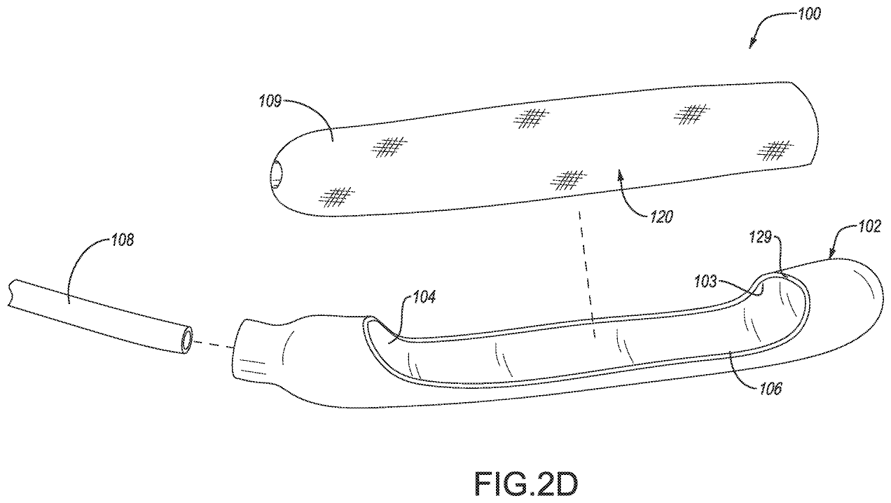

FIG. 2A is an isometric view of the fluid collection device 100, according to an embodiment. The fluid collection device 100 is an example of a female fluid collection device 100 that is configured to receive fluids from a female. The fluid collection device 100 includes a dry adhesive region 150 (shown in FIGS. 2B and 2C) disposed or otherwise positioned on a fluid impermeable barrier 102 having a first end region 125 and a second end region 127. The fluid impermeable barrier 102 may include a front region 115 positioned to orient towards the user during use and a back or rear region 117 positioned to orient away from the user during use. The fluid impermeable barrier 102 at least partially defines a chamber 104 (e.g., interior region, shown in FIG. 2D) and includes an inward border or edge 129 on the front regions 115 defining an opening 106. The opening 106 is formed in and extends longitudinally through the fluid impermeable barrier 102, thereby enabling fluids to enter the chamber 104 from outside of the fluid collection device 100. The opening 106 may be configured to be positioned adjacent to the opening of a female urethra and the dry adhesive region 150 may be positioned at least partially (e.g., entirely) distal or opposite to the opening 106.

The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. For example, in some embodiments, the front region 115 is generally arched or rounded and the back region 117 is generally planar. According to various embodiments, the fluid impermeable barrier 102 may include any of a number of different shapes and configurations, such as the shapes and configurations of the fluid impermeable barriers disclosed in U.S. Patent Application No. 63/171,165 filed on Apr. 6, 2021, U.S. Patent Application No. 63/228,258 filed on Aug. 2, 2021, U.S. Patent Application No. 63/228,252 filed on Aug. 2, 2021, U.S. Patent Application No. 63/228,244 filed on Aug. 2, 2021, U.S. patent application Ser. No. 17/394,055 filed Aug. 4, 2021, or PCT Application No. PCT/US20/40860, the disclosures of each of which are incorporated herein by reference in their entirety. In each of the various shapes and configurations of the fluid impermeable barrier, the dry adhesive region 150 may be positioned at least partially (e.g., entirely) distal or opposite to the opening in the fluid impermeable barrier.

The fluid collection device 100 may be positioned at least proximate to the opening of the female urethra or over the urethra of a male having a buried penis, and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIG. 2C) of void space formed within the chamber 104 and/or towards the inlet 110 of the conduit 108. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 2D) of the fluid permeable body 120 interfaces with an inner surface 103 (shown in FIG. 2D) of the fluid impermeable barrier 106.

In some embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane 111 covering or wrapping around at least a portion of a fluid permeable support 113, with both the fluid permeable membrane 111 and the fluid permeable support 113 being disposed in the chamber 104. The fluid permeable membrane 111 may cover or extend across at least a portion (e.g., all) of the opening 106. The fluid permeable membrane 111 may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” In some embodiments, at least one of the fluid permeable membrane 111 or the fluid permeable support 113 include nylon configured to wick fluid away from the opening 106. The material of the fluid permeable membrane 111 and the fluid permeable support 113 also may include natural fibers. In such examples, the material may have a coating to prevent or limit absorption of fluid into the material, such as a water repellent coating. Such “wicking” or other physical properties may not include absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption is desired, the term “substantially no absorption” may allow for nominal amounts of absorption of fluid into the wicking material (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 120. In an embodiment, the fluid permeable body 120 may include at least one absorbent or adsorbent material.

The fluid permeable membrane 111 may also wick the fluid generally towards an interior of the chamber 104, as discussed in more detail below. The fluid permeable membrane 111 may include any material that may wick the fluid. For example, the fluid permeable membrane 111 may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane 111 from gauze, soft fabric, and/or smooth fabric may reduce chaffing caused by the fluid collection device 100. Other embodiments of fluid permeable membranes 111, fluid permeable supports 113, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support 113 including a porous nylon structure (e.g., spun nylon fibers) and a fluid permeable membrane 111 including gauze about or over the porous nylon structure of the fluid permeable support 113.

In use, the fluid permeable body 120 of the fluid collection device is positioned adjacent to a urethra of the user. The fluid permeable body 120 is disposed within a chamber 104 (shown in FIG. 2D) of the fluid impermeable barrier 102 of the fluid collection device 100 and is exposed to the urethra of the user through the opening 106 in the fluid collection device 100. The fluid collection device 100 may be secured to the user with any of a number of securing devices. Fluids received in the chamber 104 of the fluid collection device 100 from the urethra may be removed through the conduit 108.

FIG. 2B illustrates a rear view of the fluid collection device 100 including the dry adhesive region 150 positioned on the rear region 117 of the fluid impermeable barrier 102, according to an embodiment. As noted above, the fluid collection device 100 may be positioned at least proximate to the opening of the female urethra or over the urethra of a male having a buried penis, and urine may enter the interior region of the fluid collection device 100 via the opening 106. In this position, the dry adhesive region 150 is positioned on the fluid collection device 100 to orient towards and/or interface (e.g., directly contact) the garment 20 (e.g., an undergarment) worn by the user over the fluid collection device 100. In some embodiments, the dry adhesive region 150 is integral with the fluid impermeable barrier 102. For example, the dry adhesive region 150 may be molded with the fluid impermeable barrier 102. In some embodiments, the dry adhesive region 150 is formed on (e.g., integrally formed with) at least a portion of the rear region 115 with laser lithography. In some embodiments, the dry adhesive region 150 is secured or securable to at least a portion of the rear region 115, such as after the fluid impermeable barrier 102 has been formed. In these and other embodiments, the dry adhesive region 150 may be secured to the fluid impermeable barrier with at least one of an adhesive or welding (e.g., ultrasonic welding or radio frequency welding). For example, the dry adhesive region 150 may include a patch having the dry adhesive region 150 on a substrate that is secured or securable to the rear region 117 of the fluid impermeable barrier 102. The dry adhesive region 150 may include any of the materials described in relation to the fluid impermeable barrier 102. In some embodiments, the dry adhesive region 150 may include one or more of silicone or polyurethane.

In some embodiments, such as embodiments including a generally cylindrical fluid impermeable barrier 102, the dry adhesive region 150 may exhibit an elongated shape. For example, the dry adhesive region 150 may extend longitudinally along at least a portion of the rear region 115 of the fluid impermeable barrier 102. The dry adhesive region 150 on the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The dry adhesive region 150 may exhibit a width that is greater than 50% of the circumference of the fluid collection device 100. In some embodiments, the dry adhesive region 150 may be vertically oriented (e.g., having a major axis parallel to the longitudinal axis of the device 100). In some embodiments, (not shown), the dry adhesive region 150 may be horizontally oriented (e.g., having a major axis perpendicular to the longitudinal axis of the device 100).

In some embodiments, the dry adhesive region 150 extends across and/or covers at least some of the rear region 115 of the fluid impermeable barrier 102, such as at least about 10% of the surface area of the rear region 115, at least about 25% of the surface area of the rear region 115, at least about 50% of the surface area of the rear region 115, at least about 75% of the surface area of the rear region 115, at least about 90% of the surface area of the rear region 115, less than about 10% of the surface area of the rear region 115, less than about 25% of the surface area of the rear region 115, less than about 50% of the surface area of the rear region 115, less than about 75% of the surface area of the rear region 115, less than about 90% of the surface area of the rear region 115, about 1% to about 10% of the surface area of the rear region 115, about 10% to about 25% of the surface area of the rear region 115, about 25% to about 50% of the surface area of the rear region 115, about 50% to about 75% of the surface area of the rear region 115, about 75% to about 90% of the surface area of the rear region 115, or about 90% to about 100% of the surface area of the rear region 115 of the fluid impermeable barrier 102.

In at least one, some, or all embodiments, the dry adhesive region 150 provides one or more technical effects including allowing the fluid collection device 100 to stick to the inside of the garment 20 for improved placement, comfort, and/or securement of the fluid collection device 100, especially during sleep. Conventional fluid collection devices are easily knocked out of place, and traditional wet adhesives requiring drying/curing and/or may damage garments. The dry adhesive region 150 may increase surface friction and promote shear adhesion effective to anchor to the interstices of the garment 20 by the generation of Van der Waals forces.

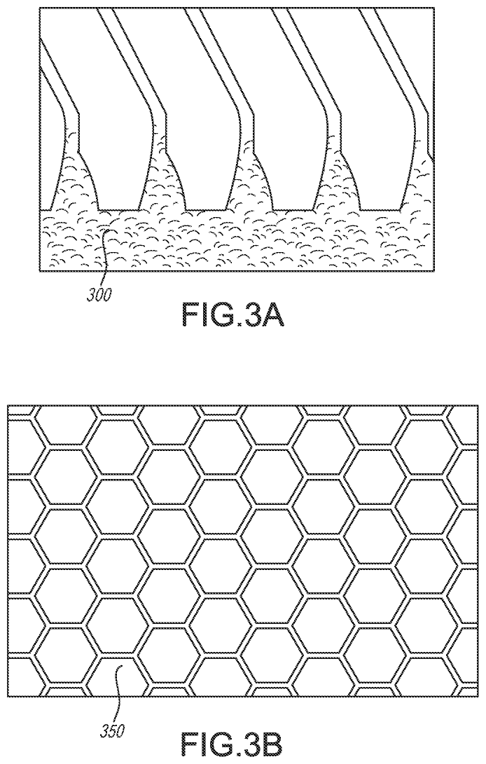

In some embodiments, the dry adhesive region 150 may include biomimetic synthetic gecko material. For example, the dry adhesive region may include micro or nanometer-scaled polymer brushes or hairs on the dry adhesive region 150. Turning to FIG. 3A, an embodiment of a dry adhesive region 300 may include wall-shaped hierarchical micro or nanometer-scaled structures that result in the technical effect of a gecko-like attachment of the fluid collection device 100 to the garment 20. In some embodiments, the dry adhesive region 150 may include micro or nanometer-scaled patterning by, for example, lithography or lasers. The micro or nanometer-scaled patterning may include geometrical shapes, such as circular, triangular, rectangular, pentagonal, hexagonal, heptagonal, octagonal, etc., shapes. For example, turning to FIG. 3B, an embodiment of a dry adhesive region 350 may include micro or nanometer-scaled patterning having hexagonal shapes formed on a substrate which increases the roughness of the substrate, resulting in the technical effect of improving friction adhesion to the fabric of the garment 20. The micro or nanometer-scaled patterning may include dimples, such as circular, triangular, rectangular, pentagonal, hexagonal, heptagonal, octagonal, etc., dimples. In some embodiments, the micro or nanometer-scaled patterning may include three-dimensional fibrillar structures on the surface of the fluid impermeable barrier 102 or other substrate (e.g., polymer substrate). In some embodiments, the dry adhesive region 150 includes one or more of: a biomimetic dry adhesive region; micro or nanometer-scaled wedges; hierarchical polymer micro or nanometer-scaled hairs; wall-shaped hierarchical micro or nanometer-scaled structures; one or more micro or nanometer-scaled fibrillar structures; micro or nanometer-scaled brushes; micro or nanometer-scaled mushroom shaped structures; micro or nanometer-scaled patterned material having polygonal or round patterns, round or polygonal dimples, and/or polygonal or round pillars; and/or micro or nanometer-scaled tubes. In some embodiments, the dry adhesive region 150 includes micro or nanometer scaled polymer brushes or hairs integrally formed with or secured to the fluid impermeable barrier 102. In some embodiments, the dry adhesive region includes micro or nanometer scaled patterning of a geometrical shape (e.g., hexagon) or three-dimensional fibrillary structures directly on the rear region 117 of the fluid impermeable barrier 102 or on a patch secured to the rear region 117 of the fluid impermeable barrier 102.

Other embodiments of materials, structures, and methods of forming particular dry adhesive regions that may be used on the dry adhesive region 150 are disclosed in U.S. Pat. No. 8,703,032 filed on Oct. 14, 2010; “A microfabricated wedge-shaped adhesive array displaying gecko-like dynamic adhesion, directionality,” J.R. Soc. Interface (2009) 6, 1223-1232; “A nontransferring dry adhesive with hierarchical polymer nanohairs,” PNAS (2009) vol. 6, no. 104, 5639-5644; “Adhesion, friction, and compliance of bio-mimetic and bio-inspired structured interfaces,” Materials Science and Engineering R (2011) 72, 253-292; “Bioinspired dry adhesive: Poly(dimethylsiloxane) grafted with poly(2-ethylhexyl acrylate) brushes,” European Polymer Journal (2015) 68, 432-440; “Continuous Fabrication of Wide-Tip Microstructures for Bio-Inspired Dry Adhesives via Tip Ink Process,” Hindawi Journal of Chemistry (2019) Article ID 4827918, 5 pages; “Dry solution to a sticky problem,” Nature (2012) 477(7362): 42-43; “Effect of nano- and micro-roughness on adhesion of bioinspired micropatterned surfaces,” Acta Biomaterialia (2012) 8, 282-288; Frictional adhesion: a new angle on gecko attachment,” The Journal of Experimental Biology (2006) 209, 3569-3579; “Multifunctional Smart Skin Adhesive Patches for Advanced Health Care,” Advanced Healthcare Materials (2018) 7, 1800275; “Nanobiodiversity and Biomimetic Adhesives Development: From Nature to Production and Application,” Journal of Biomaterials and Nanobiotechnology (2019) 10, 78-101; Nanohairs and nanotubes: Efficient structural elements for gecko-inspired artificial dry adhesives,” Nano Today (2009) 4, 335-346; “Non-sticky and Non-slippery Biomimetic Patterned Surfaces,” Journal of Bionic Engineering (2020) 17, 326-334; “Use of biomimetic hexagonal surface texture in friction against lubricated skin,” J. R. Soc. Interface (2014) 11: 20140113, the disclosure of each of which is incorporated herein, in its entirety, by this reference.

FIG. 2C is a cross-sectional view of the fluid collection device 100 taken along line 2-2 of FIG. 2A. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 2C, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 2C. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, an end of the conduit 108 may be generally flush or coplanar with the second body end 123 of the fluid permeable body 120. In other embodiments, the end of the conduit 108 may be recessed from the second body end 123 of the fluid permeable body 120. The end of the conduit 108 also may be selectively moveable between partially extending into the reservoir 122 (shown in FIG. 2C) and recessed from or flush with the second body end 123 of the fluid permeable body (not shown).

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 2A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

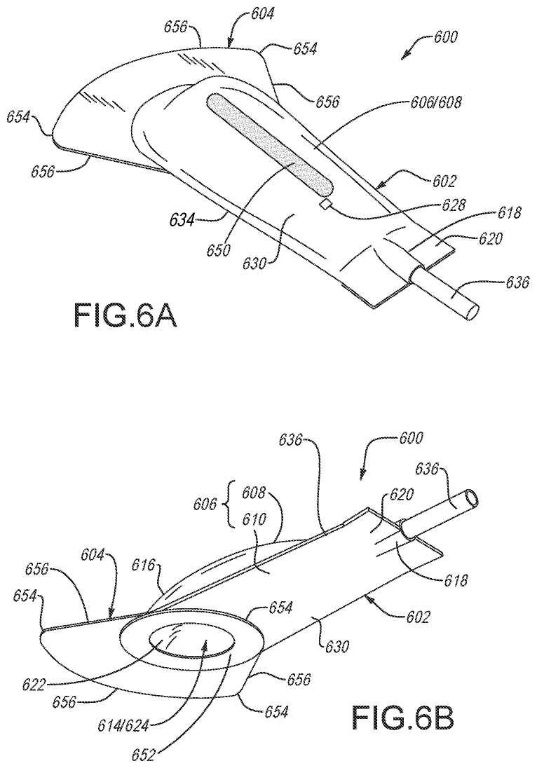

In some embodiments, fluid collection devices configured for use with a penis on a male may include any of the dry adhesive regions and materials described herein. Turning ahead in the drawings to FIGS. 6A and 6B, isometric top and bottom views, respectively, of a fluid collection device 600 having a dry adhesive region 650 are illustrated, according to an embodiment. The fluid collection device 600 includes a sheath 602 and a base 604. The sheath 602 includes a fluid impermeable barrier 606 that is at least partially formed from a first panel 608 attached to a second panel 610. In an embodiment, as illustrated, the first panel 608 and the second panel 610 are distinct sheets. In some embodiments, the first panel 608 and the second panel 610 are integral with one another. The fluid impermeable barrier 606 also defines a chamber between the first panel 608 and the second panel 610, an opening 614 at a proximal end region 616 of the sheath 602, and an outlet 618 at a distal end region 620 of the sheath 602. The sheath 602 also includes at least one porous material 622 disposed in the chamber. The base 604 includes an aperture 624. The base 604 is permanently attached to the proximal end region 616 of the sheath 602 such that the aperture 624 is aligned with the opening 614. Permanently attached means that the sheath 602 cannot be detached from the base 604 without damaging at least one of the sheath 602 or the base 604, using a blade to separate the sheath 602 from the base 604, and/or using chemicals to dissolve the adhesive that attaches the sheath 602 from the base 604.

The dry adhesive region 650 is positioned on an outer surface of the first panel 608 (e.g., rear panel or rear region), opposite to opening 614 and/or the second panel 610. The dry adhesive region 650 may include any materials or configurations of the dry adhesive region 150. The dry adhesive region 650 may be secured to or formed with the first panel 608 according to any aspect of the dry adhesive region 150 being secured to or formed with the fluid impermeable barrier 102.

The dry adhesive region 650 on the first panel 608 may exhibit a width that is measured transverse to the longitudinal direction of the fluid collection device 600 and may be at least about 10% of the width of the fluid collection device 600, such as about 25% to about 50%, about 40% to about 60%, about 50% to about 75%, about 65% to about 85%, or about 75% to about 100% of the width of the fluid collection device 600. The dry adhesive region 650 may exhibit a width that is greater than 50% of the width of the fluid collection device 600. In some embodiments, the dry adhesive region 650 may be vertically oriented (e.g., having a major axis parallel to the longitudinal axis of the device 600). In some embodiments, (not shown), the dry adhesive region 650 may be horizontally oriented (e.g., having a major axis perpendicular to the longitudinal axis of the device 600).

In some embodiments, the dry adhesive region 650 extends across and/or covers at least some of the first panel 608 of the fluid impermeable barrier 102, such as at least about 10% of the surface area of the first panel 608, at least about 25% of the surface area of the first panel 608, at least about 50% of the surface area of the first panel 608, at least about 75% of the surface area of the first panel 608, at least about 90% of the surface area of the first panel 608, less than about 10% of the surface area of the first panel 608, less than about 25% of the surface area of the first panel 608, less than about 50% of the surface area of the first panel 608, less than about 75% of the surface area of the first panel 608, less than about 90% of the surface area of the first panel 608, about 1% to about 10% of the surface area of the first panel 608, about 10% to about 25% of the surface area of the first panel 608, about 25% to about 50% of the surface area of the first panel 608, about 50% to about 75% of the surface area of the first panel 608, about 75% to about 90% of the surface area of the first panel 608, or about 90% to about 100% of the surface area of the first panel 608 of the fluid impermeable barrier 606.

The inner surfaces 626 of the fluid impermeable barrier 606 (e.g., inner surfaces of the first and second panels 608, 610) at least partially defines the chamber within the fluid collection device 600. The fluid impermeable barrier 606 temporarily stores the bodily fluids in the chamber. The fluid impermeable barrier 606 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 606 substantially prevents the bodily fluids from passing through the fluid impermeable barrier 606. In an example, the fluid impermeable barrier 606 may be air permeable and fluid impermeable thus preventing leaks while allowing air flow through the chamber when a suction force is applied thereto (i.e., the chamber remains at about atmospheric pressure thereby preventing the suction force from causing a hickie or kinking the conduit 636). In such an example, the fluid impermeable barrier 606 may be formed of a hydrophobic material that defines a plurality of pores. Alternatively or additionally, the fluid impermeable barrier 606 may include at least one perforation 628 (e.g., vacuum relief hole) that allows the chamber to remain substantially at atmospheric pressure. At least one or more portions of at least an outer surface 630 of the fluid impermeable barrier 606 may be formed from a soft and/or smooth material, thereby reducing chaffing.

In an embodiment, at least one of the first panel 608 or the second panel 610 is formed from an at least partially transparent fluid impermeable material, such as polyethylene, polypropylene, polycarbonate, or polyvinyl chloride. Forming at least one of the first panel 608 or the second panel 610 from an at least partially transparent fluid impermeable material allows a person (e.g., medical practitioner) to examine the penis. In some embodiments, both the first panel 608 and the second panel 610 are formed from at least partially transparent fluid impermeable material. For example, some conventional fluid collection assemblies that include a sheath and a base may allow the sheath to be reversibly detached from the base after the base is secured to the region about the penis. Detaching the sheath from the base allows the person to examine the penis. However, configuring the sheath to be detachable from the base may allow leaks between the sheath and the base. As previously discussed, the sheath 602 is permanently attached to the base 604 which substantially prevents leaks between the sheath 602 and the base 604 when the base 604 is appropriately attached to the sheath 602 (e.g., no wrinkles were allowed to form between the sheath 602 and base 604). Selecting at least one of the first panel 608 or the second panel 610 to be formed from an at least partially transparent impermeable material allows the penis to be examined without detaching the entire fluid collection device 600 from the region about the penis. For example, the chamber may include a penis receiving area 632 that is configured to receive the penis of the individual when the penis extends into the chamber. The penis receiving area 632 may be defined by at least the porous material 622 and at least a portion of the at least partially transparent material of the first panel 608 and/or the second panel 610. In other words, the porous material 622 is positioned in the chamber such that the porous material is not positioned between the penis and at least a portion of the transparent portion of the first panel 608 and/or second panel 610 when the penis is inserted into the chamber through the opening 614. The porous material 622 is generally not transparent and, thus, the portion of the at least partially transparent material of the first panel 608 and/or the second panel 610 that defines the penis receiving area 632 forms a window which allows the person to view into the penis receiving area 632 and examine the penis.

In an embodiment, the second panel 610 is at least partially formed from the at least partially transparent material and forms the window that allows the person to view into the penis receiving area 632. Further, the porous material 622 is positioned between the penis receiving area 632 and at least a portion of the first panel 608. Such an embodiment may help maintain the dignity of the individual using the fluid collection device 600. For example, during use, the second panel 610 is generally adjacent to the individual, such as adjacent to the thighs and/or perineum. Thus, the second panel 610 is generally obscured during use and a person cannot view the penis without first lifting the sheath 602 away from the individual. Meanwhile, the first panel 608 may face away from the individual and be more easily viewable than the second panel 610. However, a person (e.g., a passerby, a visitor, etc.) cannot view the penis through the first panel 608 because the porous material 622 is not transparent and/or the first panel 608 is formed from a non-transparent material. Thus, in such an embodiment, the first panel 608 and/or the porous material 622 prevent person(s) from viewing the penis unless such examination is necessary, thereby preserving the dignity of the individual using the fluid collection device 600. In an embodiment, the first panel 608 is formed from the at least partially transparent material and forms the window that allows the person to view into the penis receiving area 632. Further, the porous material 622 is positioned between the penis receiving area 632 and at least a portion of the second panel 610. In such an embodiment, the person does not need to perform the additional act of lifting the sheath 602 to view into the penis receiving area 632 but may not maintain the dignity of the individual using the fluid collection device 600 since passersby may also view into the penis receiving area 632.

As previously discussed, at least a portion of the first panel 608 and at least a portion of the second panel 610 are attached together. In an embodiment, as shown, the first and second panels 608, 610 are attached together along at least a portion of the outer edges 634 thereof. In such an embodiment, the first and second panels 608, 610 are attached using any suitable technique, such as with an adhesive, sewing, heat sealing, radio frequency (“RF”) welding, ultrasonic (“US”) welding, or any other technique. As will be discussed in more detail below, forming the fluid impermeable barrier 606 from the first panel 608 and the second panel 610 may improve the rate of manufacturing the fluid collection device 600, especially when the first panel 608 and the second panel 610 are attached together using a non-sewing technique.

The opening 614 defined by the fluid impermeable barrier 606 provides an ingress route for fluids to enter the chamber when the penis is a buried penis and allow the penis to enter the chamber (e.g., the penis receiving area 632) when the penis is not buried. The opening 614 may be defined by the fluid impermeable barrier 606 (e.g., an inner edge of the fluid impermeable barrier 606). For example, the opening 614 is formed in and extends through the fluid impermeable barrier 606, from the outer surface 630 to the inner surface 626, thereby enabling bodily fluids to enter the chamber from outside of the fluid collection device 600.

In the illustrated embodiment, the second panel 610 defines the entirety of the opening 614. For example, the opening 614 is a cutout defined by the second panel 610 that is spaced from the outer edges 634 of the second panel 610. In such an example, the second panel 610 may exhibit a shape that substantially corresponds to the shape of the first panel 608 which may facilitate attaching the first panel 608 to the second panel 610 along the outer edges thereof. It also allows the first panel 608 and the second panel 610 to lie substantially flat when the penis is not in the chamber which may make wearing the fluid collection device 600 more discrete and inhibit pooling of bodily fluids against the individual. However, in some embodiments, the opening 614 is not spaced from the outer edges 634 of the second panel 610. In such embodiments, the opening 614 may be a cutout extending inwardly from at least one outer edge 634 of the second panel 610. In some embodiments, the opening 614 may not be formed as a cutout.

Referring back to FIGS. 6A-6B, the fluid impermeable barrier 606 defines an outlet 618 sized to receive a conduit 636. The conduit 636 may be at least partially disposed in the chamber or otherwise in fluid communication with the chamber through the outlet 618. The outlet 618 may be sized and shaped to form an at least substantially fluid tight seal against the conduit 636 thereby substantially preventing the bodily fluids from escaping the chamber. In an embodiment, the outlet 618 may be formed from a portion of the first panel 608 and the second panel 610 that are not attached together. In such an embodiment, the fluid impermeable barrier 606 may not include a cap exhibiting a rigidity that is greater than the portions of the fluid impermeable barrier 606 thereabout which may facilitate manufacturing of the fluid collection device 600 may decreasing the number of parts that are used to form the fluid collection device 600 and may decrease the time required to manufacture the fluid collection device 600. The lack of the cap may make securing the conduit 636 to the outlet 618 using interference fit to be difficult though, it is noted, attaching the conduit 636 to the outlet 618 may still be possible. As such, the conduit 636 may be attached to the outlet 618 (e.g., to the first and second panels 608, 610) using an adhesive, a weld, or otherwise bonding the outlet 618 to the outlet 618. Attaching the conduit 636 to the outlet 618 may prevent leaks and may prevent the conduit 636 from inadvertently becoming detached from the outlet 618. In an example, the conduit 636 may be attached to the outlet 618 in the same manufacturing step that attaches the first and second panels 608, 610 together.

As previously discussed, the sheath 602 includes at least one porous material 622 disclosed in the chamber. The porous material 622 may direct the bodily fluids to one or more selected regions of the chamber, such as away from the penis and towards the outlet 618. As such, the porous material 622 may facilitate the removal of the bodily fluids from the chamber and form a padding layer that prevents the penis from resting against a damp material which may cause degradation of the skin of the penis and/or make the fluid collection device 600 more uncomfortable to wear. The porous material 622 may also blunt a stream of urine from the penis.

In an embodiment, the porous material 622 is a wicking material configured to wick any bodily fluids away from the opening 614 thereby preventing the bodily fluids from escaping the chamber. The porous material 622 may include any material and configurations (e.g., layers) of the fluid permeable body 120 described above. For example, the porous material 622 may include a first layer and a second layer. The first and second layers may be a woven material. The porous material 622 also includes a plurality of fibers forming a layer between the first layer and the second layer. Each of the first layer 638, the second layer, and the plurality of fibers define a plurality of pores, thereby allowing transport of the bodily fluids and air circulation through the porous material 622. The pores defined by the plurality of fibers may be at least one of larger or more numerous, thereby decreasing the likelihood that dried bodily fluids clog the porous material 622. The presence of the plurality of fibers also cause the porous material 622 feel soft against the penis and provides a cushioning effect to the penis. The plurality of fibers may also prevent the suction force from collapsing the porous material.

The first layer, the second layer, and the plurality of fibers may be formed from any suitable material, such as a hydrophobic material, a hydrophilic material, polyester, cotton, or any other porous material disclosed herein. In an embodiment, one or more of the first layer, the second layer, or the plurality of fibers are formed from a hydrophobic material that inhibits the porous material 622 from storing the bodily fluids therein which may facilitate removal of the bodily fluids from the chamber. In an embodiment, one or more of the first layer, and second layer, or the plurality of fibers are formed from a hydrophilic material which allows the porous material 622 to temporarily store the bodily fluids therein thereby limiting the quantity of bodily fluids that pool around the skin of the individual. In an embodiment, two or more of the first layer, the second layer, or the plurality of fibers are formed from different materials. In such an embodiment, the first layer may define the penis receiving area or is otherwise closer to the penis receiving area than the second layer. The first layer may be formed from a hydrophobic material while the plurality of fibers are formed from a hydrophilic material. Such a configuration may cause the bodily fluids to be pulled through the first layer and temporarily stored in the plurality of fibers. However, the first layer may remain substantially dry due to the hydrophobicity thereof which allows the porous material 622 to feel dry to the penis.

In an embodiment, not shown, the porous material 622 may be formed from two layers instead of three layers. For example, the porous material 622 may be formed from a fluid permeable membrane and a fluid permeable support. The fluid permeable support may define or otherwise be closer to the penis receiving area 632 than the fluid permeable support. The fluid permeable membrane may be composed and/or structured to wick bodily fluids away from the penis receiving area, thereby minimizing the quantity of bodily fluids that are present in the penis receiving area or otherwise present against the skin of the individual. It is also noted that the fluid permeable membrane may also be configured to adsorb or absorb the bodily fluids to minimize the quantity of bodily fluids that are present in the penis receiving area or otherwise present against the skin of the individual. The fluid permeable membrane may be formed from any of the porous materials disclosed herein. For example, the fluid permeable membrane may be formed from fabric, such as a gauze (e.g., silk, linen, or cotton gauze), another soft fabric, or another smooth fabric. Forming the fluid permeable membrane from gauze, soft fabric, and/or smooth fabric (or any of the other porous materials 622 disclosed herein that may contact the penis) may reduce chaffing caused by the fluid collection device 600.

The fluid permeable support is configured to support the fluid permeable membrane since the fluid permeable membrane may be formed from a relatively foldable, flimsy, or otherwise easily deformable material. For example, the fluid permeable support may be positioned such that the fluid permeable membrane is disposed between the fluid permeable support and the fluid impermeable barrier 606. As such, the fluid permeable support may support and maintain the position of the fluid permeable membrane. The fluid permeable support may include any of the fluid permeable membrane materials disclosed herein above. For example, the fluid permeable membrane material(s) may be utilized in a more dense or rigid form than in the fluid permeable membrane when used as the fluid permeable support. The fluid permeable support may be formed from any fluid permeable material that is less deformable than the fluid permeable membrane. For example, the fluid permeable support may include a porous polymer (e.g., nylon, polyester, polyurethane, polyethylene, polypropylene, etc.) structure or an open cell foam. In some examples, the fluid permeable support may be formed from a natural material, such as cotton, wool, silk, or combinations thereof. In such examples, the material may have a coating to prevent or limit absorption of fluid into the material, such as a water repellent coating. In some examples, the fluid permeable support may be formed from fabric, felt, gauze, or combinations thereof.

In an embodiment, the porous material 622 may include a single layer (e.g., one of the first layer, the second layer, the layer formed from the plurality of fibers, the fluid permeable membrane, the fluid permeable support, or another porous layer). In an embodiment, the porous material 622 may be formed from four or more layers.

In an embodiment, the porous material 622 may be a sheet. Forming the porous material 622 as a sheet may facilitate the manufacturing of the fluid collection device 600. For example, forming the porous material 622 as a sheet allows the first panel 608, the second panel 610, and the porous material 622 to each be sheets. During the manufacturing of the fluid collection device 600, the first panel 608, the second panel 610, and the porous material 622 may be stacked and then attached to each other in the same manufacturing step. For instance, the porous material 622 may exhibit a shape that is the same size or, more preferably, slightly smaller than the size of the first panel 608 and the second panel 610. As such, attaching the first panel 608 and the second panel 610 together along the outer edges 634 thereof may also attach the porous material 622 to the first panel 608 and the second panel 610. The porous material 622 may be slightly smaller than the first panel 608 and the second panel 610 such that the first panel 608 and/or the second panel 610 extend around the porous material 622 such that the porous material 622 does not form a passageway through the fluid impermeable barrier 606 through which the bodily fluids may leak. Also, attaching the porous material 622 to the first panel 608 and/or the second panel 610 may prevent the porous material 622 from significantly moving in the chamber, such as preventing the porous material 622 from bunching together near the outlet 618. In an example, the porous material 622 may be attached to the first panel 608 or the second panel 610 (e.g., via an adhesive) before or after attaching the first panel 608 to the second panel 610. In an example, the porous material 622 may merely be disposed in the chamber without attaching the porous material 622 to at least one of the first panel 608 or the second panel 610. In an embodiment, as will be discussed in more detail below, the porous material 622 may exhibit shapes other than a sheet, such as a hollow generally cylindrical shape.

Generally, the sheath 602 is substantially flat when the penis is not in the penis receiving area 632 and the sheath 602 is resting on a flat surface. The sheath 602 is substantially flat because the fluid impermeable barrier 606 is formed from the first panel 608 and the second panel 610 instead of a generally tubular fluid impermeable barrier. Further, as previously discussed, the porous material 622 may be a sheet, which also causes the sheath 602 to be substantially flat. The sheath 602 may also be substantially flat because the fluid collection device 600 may not include relatively rigid rings or caps that exhibit a rigidity that is greater than the portions of the fluid impermeable barrier 606 thereabout since such rings and caps may inhibit the sheath 602 being substantially flat. It is noted that the sheath 602 is described as being substantially flat because at least one of the porous material 622 may cause a slight bulge to form in the sheath 602 depending on the thickness of the porous material 622, the outlet 618 and/or conduit 636 may cause a bulge thereabout, or the base 604 may pull on portions of the sheath 602 thereabout. It is also noted that the sheath 602 may also be compliant and, as such, the sheath 602 may not be substantially flat during use since, during use, the sheath 602 may rest on a non-flat surface (e.g., may rest on the testicles, the perineum, and/or between the thighs) and the sheath 602 may conform to the surface of these shapes.

The ability of the sheath 602 to be substantially flat when the penis is not in the penis receiving area 632 and the sheath 602 is resting on a flat surface allows the fluid collection device 600 to be used with a buried and a non-buried penis. For example, when the fluid collection device 600 is being used with a buried penis, the penis does not extend into the penis receiving area 632 which causes the sheath 602 to lie relatively flat across the aperture 624. When the sheath 602 lies relatively flat across the aperture 624, the porous material 622 extends across the aperture and is in close proximity to the buried penis. As such, the porous material 622 prevents or inhibits pooling of bodily fluids discharged from the buried penis against the skin of the individual since the porous material 622 will receive and remove at least a significant portion of the bodily fluids that would otherwise pool against the skin of the individual. Thus, the skin of the individual remains dry thereby improving comfort of using the fluid collection device 600 and preventing skin degradation. However, unlike other conventional fluid collection assemblies that are configured to be used with buried penises, the fluid collection device 600 may still be used with a non-buried penis since the non-buried penis can still be received into the penis receiving area, even when the penis is fully erect. Additionally, the ability of the sheath 602 to be substantially flat allows the fluid collection device 600 to be used more discretely than if the sheath 602 was not substantially flat thereby avoiding possibly embarrassing scenarios.

When the sheath 602 is substantially flat, the porous material 622 occupies substantially all of the chamber and the penis receiving area is collapsed. In other words, the sheath 602 may not define a region that is constantly unoccupied by the porous material 622. When the porous material 622 occupies substantially all of the chamber, the bodily fluids discharged into the chamber are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 608, the second panel 610, and the porous material 622 may be selected to be relatively flexible. The first panel 608, the second panel 610, and the porous material 622 are relatively flexible when the first panel 608, the second panel 610, and the porous material 622, respectively, are unable to maintain their shape when unsupported. The flexibility of the first panel 608, the second panel 610, and the porous material 622 may allow the sheath 602 to be substantially flat, as discussed above. The flexibility of the first panel 608, the second panel 610, and the porous material 622 may also allow the sheath 602 to conform to the shape of the penis even when the size and shape of the penis changes (e.g., becomes erect) and to minimize any unoccupied spaces in the chamber in which bodily fluids may pool.

As previously discussed, the fluid collection device 600 includes a base 604 that is configured to be permanently attached to the sheath 602. The base is configured to be permanently attached to the sheath 602 when, for example, when the fluid collection device 600 is provided with the base 604 permanently attached to the sheath 602 or the base 604 is provided without being permanently attached to the sheath 602 but is configured to be permanently attached to the sheath 602 at some point in the future. The base 604 may be permanently attached to the sheath 602 using any suitable technique. For example, the base 604 may be permanently attached to the sheath 602 using an adhesive, sewing, heat sealing, RF welding, or US welding.

As previously discussed, the base 604 is sized, shaped, and made of a material to be coupled to the skin that surrounds the penis (e.g., mons pubis, thighs, testicles, and/or perineum) and have the penis disposed therethrough. For example, the base 604 may define an aperture 624 configured to have the penis positioned therethrough. In an example, the base 604 may exhibit the general shape or contours of the skin surface that the base 604 is configured to be coupled with. The base 604 may be flexible, thereby allowing the base 604 to conform to any shape of the skin surface and mitigate the base 604 pulling the on skin surface. The base 604 may extend laterally past the sheath 602 thereby increasing the surface area of the skin of the individual to which the fluid collection device 600 may be attached compared to a substantially similar fluid collection device 600 that did not include a base.

In an embodiment, the base 604 is at least partially transparent (e.g., a substrate and an adhesive layer are formed from at least partially transparent materials). In such an embodiment, a person (e.g., medical practitioner) may be able to examine the skin surrounding the penis, such as to determine the health of the skin. Further, the person may be able to detect any gaps between the base 604 and the skin of the individual through which bodily fluids may leak. A person may be able to eliminate the gaps or replace the fluid collection device 600 after detecting the gaps to prevent leaks and prevent degradation of the skin caused by the skin being in contact with the bodily fluids.

In some embodiments, a ring 652 at least partially surrounds and/or at least partially defines the opening 614. At least a portion (e.g., all) of the ring 652 may be a dry adhesive ring that includes any aspect, materials, and/or configurations of the dry adhesive region 150 on an outer surface of the ring 652 that interfaces the body of the user around the penis. When the ring 652 includes the dry adhesive ring, the dry adhesive region 650 on the first panel 608 may be present or may be absent. In some embodiments, the surface of the base 604 that interfaces the body of the user also may include any aspect, materials, and/or configurations of the dry adhesive region 150. In some embodiments, the base 604 may include an adhesive layer formed from any adhesive that may safely attach the substrate of the base 604 to the skin surrounding the penis. In an example, the adhesive layer may be formed from a silicone-based adhesive, such as a silicone-gel adhesive. Silicone-based adhesives, such as Silicone Medical Silicone Tape 2475P available from 3M, has been found to secure the fluid collection device 600 to the skin surrounding the penis for at least 24 hours, even immediately after cleaning the skin surface with a wipe. In an example, the adhesive layer may be formed from an acrylic gel adhesive or a hydrogel.

In an embodiment, the base 604 may exhibit a generally partially triangular shape, as illustrated. For example, the base 604 may exhibit three apexes 654 and edges 656 extending between each of the apexes 654. The apexes 654 may be rounded to prevent the base 604 from digging into and hurting the individual. In an embodiment, the aperture 624 may be located off-center and closer to one of the apexes 654 than the other apexes 654. Such an embodiment may maximize the surface area of the skin to which the base 604 is attached, thereby reducing the likelihood that the base 604 leaks or inadvertently becomes detached from the individual. For example, the apex 654 closest the aperture 624 may be configured to be attached to the skin between the penis and the testicles and/or the testicles which has limited surface area to be attached to the base 604 and/or may be sensitive. Meanwhile, the portion of the base 604 opposite the apex 654 that is closest to the aperture 624 is configured to be attached to the mons pubis which has a large surface area and is less sensitive. Other examples of shapes that the base 604 may form are disclosed in U.S. Provisional Patent Application No. 62/967,977 filed on Jan. 30, 2020, the disclosure of which is incorporated herein, in its entirety, by this reference.

As previously discussed, the fluid collection device 600 includes a conduit 636. The conduit 636 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 636 may include silicon or latex. In some examples, the conduit 636 may include one or more portions that are resilient, such as to by having one or more of a diameter or wall thickness that allows the conduit to be flexible.

An inlet of the conduit 636 may be located at or near the distal end region 620 of the sheath 602 which is expected to be the gravimetrically low point of the chamber when worn by a user. Locating the inlet at or near the distal end region 620 of the sheath 602 enables the conduit 636 to receive more of the bodily fluids than if the inlet was located elsewhere and reduce the likelihood of pooling (e.g., pooling of the bodily fluids may cause microbe growth and foul odors). For instance, the bodily fluids in porous material 622 due to capillary forces. However, the bodily fluids may exhibit a preference to flow in the direction of gravity, especially when at least a portion of the porous material 622 is saturated with the bodily fluids. Accordingly, the inlet may be located in the fluid collection device 600 in a position expected to be the gravimetrically low point in the fluid collection device 600 when worn by a user.

In an example, the conduit 636 is configured to be at least insertable into the chamber, such as into the penis receiving area. In such an example, the conduit 636 may include one or more markers (not shown) on an exterior thereof that are located to facilitate insertion of the conduit 636 into the chamber. For example, the conduit 636 may include one or more markings thereon that are configured to prevent over or under insertion of the conduit 636. In another example, the conduit 636 may include one or more markings thereon that are configured to facilitate correct rotation of the conduit 636 relative to the chamber. The one or more markings may include a line, a dot, a sticker, or any other suitable marking.

As described in more detail below, the conduit 636 is configured to be coupled to, and at least partially extend between, one or more of the fluid storage container (not shown) and the vacuum source (not shown). In an example, the conduit 636 is configured to be directly connected to the vacuum source (not shown). In such an example, the conduit 636 may extend from the fluid impermeable barrier 606 by at least one foot, at least two feet, at least three feet, or at least six feet. In another example, the conduit 636 is configured to be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet and an outlet of the conduit 636 are configured to fluidly couple (e.g., directly or indirectly) the vacuum source (not shown) to the chamber. As the vacuum source applies a vacuum/suction in the conduit 636, the bodily fluids in the chamber may be drawn into the inlet 644 and out of the fluid collection device 600 via the conduit 636. In some examples, the conduit 636 may be frosted or opaque (e.g., black) to obscure visibility of the bodily fluids therein.

In some examples, the vacuum source may be remotely located from the fluid collection device. In such examples, the conduit 636 may be fluidly connected to the fluid storage container, which may be disposed between the vacuum source and the fluid collection device 600.

During operation, a male using the fluid collection device 600 may discharge bodily fluids (e.g., urine) into the chamber. The bodily fluids may pool or otherwise be collected in the chamber (e.g., received into the porous material 622). At least some of the bodily fluids may be pulled through the interior of the conduit 636 via the inlet 644. The bodily fluids may be drawn out of the fluid collection device 600 via the vacuum/suction provided by the vacuum source. During operation, the perforation 628 may substantially maintain the pressure in the chamber at atmospheric pressure even though bodily fluids are introduced into and subsequently removed from the chamber 622.

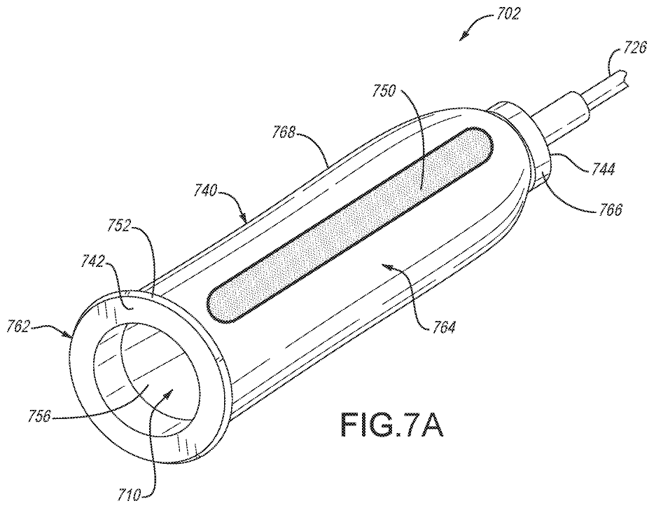

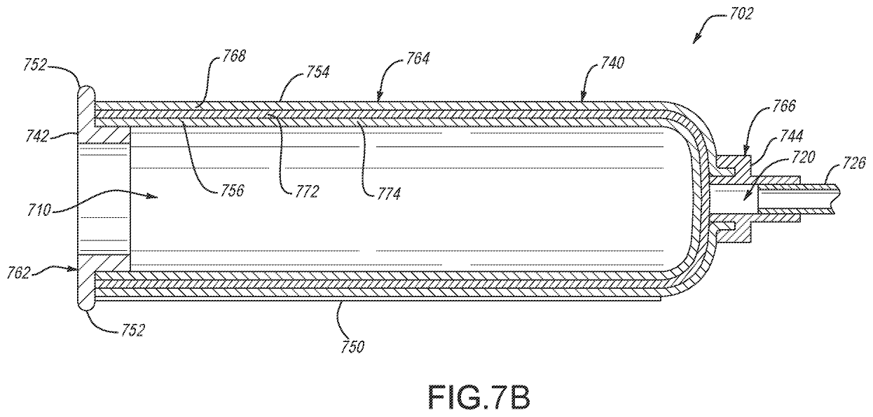

FIGS. 7A and 7B are an isometric view and a schematic cross-sectional view, respectively, of a fluid collection device 702 having a dry adhesive region 750, according to an embodiment. The fluid collection device 702 can include a body 740 having an open proximal end 742 and an at least partially closed distal end 744, a reservoir 710 at least partially defined by the body 740, and tubing 726 fluidly coupled to the reservoir 710.

The body 740 of the fluid collection device 702 can include a ring 762 at or near the open proximal end 742 of the body 740, a sheath 764 extending from or near the open proximal end 742 to or near the at least partially closed distal end 744 of the body 740, and a sump 766 at the at least partially closed distal end 744 of the body 740. In an embodiment, the ring 762, the sheath 764, and the sump 766 are all distinct components from each other. In an embodiment, at least two of the ring 763, the sheath 764, or the sump 766 are integrally formed together (e.g., are formed from a single piece).

The sheath 764 is configured to prevent a fluid (e.g., urine) escaping from the reservoir 710 and to move the fluid towards the sump 766 and the tubing 726. As such, referring to FIG. 7B, the sheath 764 can include a plurality of layers that facilitate the operation of the sheath 764. For example, the sheath 764 can include a fluid impermeable layer 768, a fluid permeable layer 774 (e.g., a one-way fluid movement fabric, gauze, or cloth), and a fluid permeable support 772 positioned between the fluid permeable layer 774 and the fluid impermeable layer 768. The fluid permeable support 772 may include a porous layer (e.g., a spun polymer layer). The fluid impermeable layer 768 can form an external surface 754 of the body 740 and prevent the fluid from leaking through the sheath 764. The fluid permeable layer 774 can form an internal surface 756 of the body 740. The fluid permeable layer 774 can be configured to move the fluid from the reservoir 710 to the fluid permeable support 772 and may substantially prevent the fluid that is in the fluid permeable support 772 from flowing back into the reservoir 710. As such, the fluid permeable layer 774 can remove fluid from around a penis thereby leaving the penis dry. The fluid permeable support 772 can form an inner layer between the fluid permeable layer 774 and the fluid impermeable layer 768. The fluid permeable support 772 can enable the fluid to flow generally towards the tubing 726.

It is noted that one or more layers of the sheath 764 can be omitted. For example, the fluid permeable layer 774 can be omitted such that the fluid permeable support 772 forms the internal surface 756 of the body 740. In such an example, the sheath 764 can rely on the wicking ability of the fluid permeable support 772 and a suction force applied to the fluid collection device 702 to remove the fluid from the penis. In another example, the sheath 764 only include the fluid impermeable layer 768. In such an example, the sheath 764 can rely on the suction force applied to the fluid collection device 702 to remove the fluid from the penis. In another example, the sheath 764 only includes the fluid impermeable layer 768 and the fluid permeable layer 774. In such an example, the sheath 764 can form a channel (not shown) between the fluid impermeable layer 768 and the fluid permeable layer 774 and the channel is fluidly coupled to the tubing 726.

The dry adhesive region 750 may include any aspect, materials, and/or configurations of the dry adhesive region 150. The dry adhesive region 750 may be secured to or formed with the body 740 according to any aspect of the dry adhesive region 150 being secured to or formed with the fluid impermeable barrier 102.

At least a portion of the dry adhesive region 750 is positioned on the external surface 754 of the body 740 to interface the garment of the user positioned over the fluid collection device 700 during use. In some embodiments, the dry adhesive region 750 is positioned on only a portion of the external surface 754 of the body 740. In some embodiments, the dry adhesive region 750 extends across substantially all of the external surface 754 of the body 754. In some embodiments, the dry adhesive region 750 may exhibit an elongated shape. For example, the dry adhesive region 750 may extend longitudinally along at least a portion of the external surface 754 of the body 740. The dry adhesive region 750 on the body 740 may exhibit a width that is measured transverse to the longitudinal direction and may be at least about 10% of the circumference of the fluid collection device 700, such as about 25% to about 50%, about 40% to about 60%, about 50% to about 75%, about 65% to about 85%, or about 75% to about 100% of the circumference of the fluid collection device 700. The dry adhesive region 750 may exhibit a width that is greater than 50% of the circumference of the fluid collection device 700. In some embodiments, the dry adhesive region 750 may be vertically oriented (e.g., having a major axis parallel to the longitudinal axis of the device 700). In some embodiments, (not shown), the dry adhesive region 750 may be horizontally oriented (e.g., having a major axis perpendicular to the longitudinal axis of the device 700).

In some embodiments, the dry adhesive region 750 extends across and/or covers at least some of the external surface 754 of the body 740, such as at least about 10% of the surface area of the external surface 754, at least about 25% of the surface area of the external surface 754, at least about 50% of the surface area of the external surface 754, at least about 75% of the surface area of the external surface 754, at least about 90% of the surface area of the external surface 754, less than about 10% of the surface area of the external surface 754, less than about 25% of the surface area of the external surface 754, less than about 50% of the surface area of the external surface 754, less than about 75% of the surface area of the external surface 754, less than about 90% of the surface area of the external surface 754, about 1% to about 10% of the surface area of the external surface 754, about 10% to about 25% of the surface area of the external surface 754, about 25% to about 50% of the surface area of the external surface 754, about 50% to about 75% of the surface area of the external surface 754, about 75% to about 90% of the surface area of the external surface 754, or about 90% to about 100% of the surface area of the external surface 754 of the body 740.

In some embodiments, at least a portion (e.g., all) of the proximal end 742 of the ring 762 may include any aspect, materials, and/or configurations of the dry adhesive region 150. When the proximal end 742 of the ring 762 includes any aspect, materials, and/or configurations of the dry adhesive region 150, the dry adhesive region 750 may be present or may be absent.

The fluid permeable layer 774 and/or the fluid permeable support 772 may include permeable material designed to wick or pass fluid therethrough. The fluid permeable layer 774 and/or the fluid permeable support 772 may include any materials described in relation to the fluid permeable body 120.

The sheath 764 is configured to have a penis disposed therein. To facilitate fluid collection and improve comfort, the sheath 764 can be flexible thereby allowing the sheath 764 to correspond to the shape of a penis. For example, the flexible sheath 764 can at least partially collapse when the penis is not erect and at least partially expand and bend to the shape of the penis as the penis becomes erect. Forming the layers of the sheath 764 from at least one of thin layers (e.g., less than 700 μm thick, and more particularly less than 250 μm thick, less than 100 μm thick, or less than 70 μm thick), flexible layers, or fabric can allow the sheath 764 to be sufficiently flexible.

The ring 762 can be more rigid than the sheath 764. For example, the ring 762 can be formed from a flexible polymer that is at least one of thicker than the entire sheath 764 or exhibits a Young's modulus that is greater than sheath 764. As such, the ring 762 can provide some structure at or near the open proximal end 742 of the body 740. The increased rigidity of the ring 762 can cause the open proximal end 742 to remain open thereby facilitating insertion of a penis into the urine collecting assembly 702. Further, in an embodiment, the increased rigidity of the ring 762 can enable the ring 762 to act as an attachment mechanism. For example, as illustrated, the ring 762 can include at least one protrusion 752 that extends from the rest of the body 740. In another example, the ring 762 can define a recess, include threads, or include any other attachment mechanism disclosed herein.

The sump 766 is configured to attach the rest of the fluid collection device 702 to the tube 726. For example, the sump 766 can define an outlet 720 extending through at least the fluid impermeable layer 768 thereby coupling the tubing 726 to the fluid permeable support 772 and/or the reservoir 710. Further, the sump 766 can close the at least partially closed distal end 744 of the body 740. For example, the sump 766 can bunch up the sheath 764 and close any gaps that may form.

The ring 762, the sheath 764, the sump 766, and the tubing 726 can be attached together using any suitable method. For example, at least two of the ring 762, the sheath 764, the sump 766, or the tubing 726 can be attached together using at least one of an interference fit, an adhesive, stitching, welding (e.g., ultrasonic welding), tape, any other suitable method, or combinations thereof.

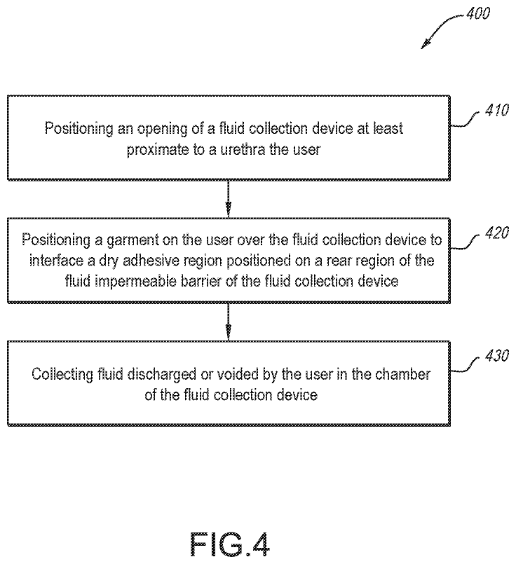

Turning back to FIG. 4, a flow diagram of a method 400 to collect fluid is provided, according to an embodiment. The method 400 can include act 410 of positioning an opening of a fluid collection device at least proximate to a urethra of the user. The fluid collection device may include any fluid collection device described herein. In some embodiments, the fluid collection device of the method 400 includes a fluid impermeable barrier that defines a chamber, the opening having fluid communication with the chamber, and an aperture having a conduit extending therethrough in fluid communication with the chamber. The method 400 includes an act 420 of positioning a garment on the user over the fluid collection device with a dry adhesive region positioned on a rear region of the fluid impermeable barrier at least partially distal to the opening to interface the garment. The dry adhesive region of the method 400 may include any aspect of the dry adhesive region(s) 150, 650, 750 described above. The method 400 includes an act 430 of collecting fluid discharged or voided by the user in the chamber of the fluid collection device.

In some embodiments, the method 400 may optionally comprise securing the dry adhesive region to the rear region of the fluid impermeable barrier. The dry adhesive region of the method 400 may include any dry adhesive region described herein. In some embodiments, the dry adhesive region of the method 400 includes one or more of: a biomimetic dry adhesive region; micro or nanometer-scaled wedges; hierarchical polymer micro or nanometer-scaled hairs; wall-shaped hierarchical micro or nanometer-scaled structures; one or more micro or nanometer-scaled fibrillar structures; micro or nanometer-scaled brushes; micro or nanometer-scaled mushroom shaped structures; micro or nanometer-scaled patterned material having polygonal or round patterns, round or polygonal dimples, and/or polygonal or round pillars; and/or micro or nanometer-scaled tubes. The fluid collection device may include a generally cylindrical shape such that the front region and the rear region of the fluid impermeable barrier are generally arched and the dry adhesive region arcs complementary to the rear region. In some embodiments, the rear region of the fluid collection may be generally planar such that the dry adhesive region is generally planar.

Acts 410, 420, 430 of the method 400 are for illustrative purposes. For example, the acts 410, 420, 430 of the method 400 can be performed in different orders, split into multiple acts, modified, supplemented, or combined. In an example, one or more of the acts 410, 420, 430 of the method 400 can be omitted from the method 400. Any of the acts 410, 420, or 430 can include using any of the dry adhesive regions, garments, fluid collection devices, vacuum sources, fluid storage containers, systems, or components of the same disclosed herein.

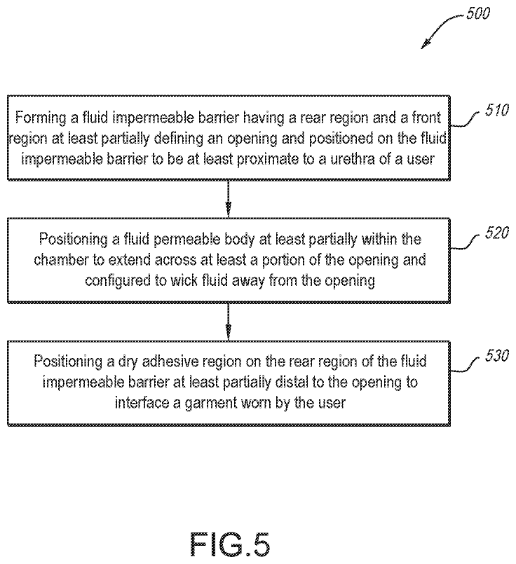

FIG. 5 is a flow diagram of a method 500 of forming a fluid collection device, according to an embodiment. The method 500 can include act 510 of forming a fluid impermeable barrier having a rear region and a front region at least partially defining an opening and positioned on the fluid impermeable barrier to be at least proximate to a urethra of a user. The fluid impermeable barrier may at least partially define a chamber and an aperture sized and dimensioned to receive a conduit therethrough. The method 500 includes an act 520 of positioning a fluid permeable body at least partially within the chamber to extend across at least a portion of the opening and configured to wick fluid away from the opening. The method 500 includes an act 530 of positioning a dry adhesive region on the rear region of the fluid impermeable barrier at least partially distal to the opening to interface a garment worn by the user. The dry adhesive region of the method 500 may include any aspect of the dry adhesive region(s) 150, 650, 750 described above.

In some embodiments, the method 500 may optionally include an act of micro or nanopatterning geometric shapes with lithography or laser to form the dry adhesive region. In these and other embodiments, positioning the dry adhesive region on the rear region of the fluid impermeable barrier may include securing a patch including the dry adhesive region on the rear region of the fluid impermeable barrier after micro or nanopatterning geometric shapes on the patch to form the dry adhesive region. In some embodiments, securing the patch on the rear region of the fluid impermeable barrier may include securing the patch on the rear region of the fluid impermeable barrier with at least one of welding or an adhesive. In some embodiments, micro or nanopatterning the geometric shapes to form the dry adhesive region may include micro or nanopatterning the geometric shapes directly on the rear region of the fluid impermeable barrier to form the dry adhesive region on the rear region of the fluid impermeable barrier.

In some embodiments, positioning a dry adhesive region on the rear region of the fluid impermeable barrier includes securing a patch including the dry adhesive region on the rear region of the fluid impermeable barrier. Securing a patch including the dry adhesive region on the rear region of the fluid impermeable barrier may include securing the patch including the dry adhesive region on the rear region of the fluid impermeable barrier with welding or an adhesive. In some embodiments, positioning a dry adhesive region on the rear region of the fluid impermeable barrier includes integrally forming the dry adhesive region directly on the rear region of the fluid impermeable barrier. The dry adhesive region may include any of the dry adhesive regions described herein such as one or more of: a biomimetic dry adhesive region; micro or nanometer-scaled wedges; hierarchical polymer micro or nanometer-scaled hairs; wall-shaped hierarchical micro or nanometer-scaled structures; one or more micro or nanometer-scaled fibrillar structures; micro or nanometer-scaled brushes; micro or nanometer-scaled mushroom shaped structures; micro or nanometer-scaled patterned material having polygonal or round patterns, round or polygonal dimples, and/or polygonal or round pillars; and/or micro or nanometer-scaled tubes.

In some embodiments of the method 500, the act 510 of forming a fluid impermeable barrier includes forming the fluid impermeable barrier having a generally cylindrical shape such that the front region and the rear region of the fluid impermeable barrier are generally arched and the dry adhesive region arcs complementary to the rear region after positioning the dry adhesive region on the rear region of the fluid impermeable barrier. The act 510 of forming a fluid impermeable barrier may include forming the fluid impermeable barrier with the rear region of being generally planar such that the dry adhesive region is generally planar after positioning the dry adhesive region on the rear region of the fluid impermeable barrier.

Acts 510, 520, 530 of the method 500 are for illustrative purposes. For example, the act 510, 520, 530 of the method 500 can be performed in different orders, split into multiple acts, modified, supplemented, or combined. In an example, one or more of the acts 510, 520, 530 of the method 500 can be omitted from the method 500. For example, the method 500 may not include the act 510. Any of the acts 510, 520, or 530 can include using any of the dry adhesive regions, garments, fluid collection devices, vacuum sources, fluid storage containers, systems, or components of the same disclosed herein.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A fluid collection device, comprising:
a fluid impermeable barrier having a rear region and a front region at least partially defining an opening and positioned on the fluid impermeable barrier to be at least proximate to a urethra of a user, the fluid impermeable barrier further at least partially defining a chamber and an aperture sized and dimensioned to receive a conduit therethrough;
a fluid permeable body positioned at least partially within the chamber to extend across at least a portion of the opening and configured to wick fluid away from the opening; and
a dry adhesive region positioned on the rear region of the fluid impermeable barrier at least partially distal to the opening to interface a garment worn by the user.

2. The fluid collection device of claim 1, wherein the dry adhesive region includes a biomimetic dry adhesive region.

3. The fluid collection device of claim 1, wherein the dry adhesive region includes at least micro or nanometer-scaled wedges.

4. The fluid collection device of claim 1, wherein the dry adhesive region includes at least hierarchical polymer micro or nanometer-scaled hairs.

5. The fluid collection device of claim 1, wherein the dry adhesive region includes at least wall-shaped hierarchical micro or nanometer-scaled structures.

6. The fluid collection device of claim 1, wherein the dry adhesive region includes at least one or more micro or nanometer-scaled fibrillar structures.

7. The fluid collection device of claim 1, wherein the dry adhesive region includes at least micro or nanometer-scaled brushes.

8. The fluid collection device of claim 1, wherein the dry adhesive region includes micro or nanometer-scaled mushroom shaped structures.

9. The fluid collection device of claim 1, wherein the dry adhesive region includes micro-scaled patterned material or nanometer-scaled patterned material.

10. The fluid collection device of claim 9, wherein the micro-scaled patterned material or the nanometer-scaled patterned material includes polygonal or round patterns, round or polygonal dimples, and/or polygonal or round pillars.

11. The fluid collection device of claim 1, wherein the dry adhesive region includes micro or nanometer-scaled tubes.

12. The fluid collection device of claim 1, wherein the dry adhesive region is a dry adhesive patch secured to the fluid impermeable barrier.

13. The fluid collection device of claim 12, wherein the dry adhesive patch is secured to the fluid impermeable barrier with at least one of an adhesive or welding.

14. The fluid collection device of claim 1, wherein the dry adhesive region is integrally formed with the fluid impermeable barrier.

15. The fluid collection device of claim 1, wherein the fluid collection device includes a generally cylindrical shape such that the front region and the rear region of the fluid impermeable barrier are generally arched and the dry adhesive region arcs complementary to the rear region.

16. The fluid collection device of claim 1, wherein the rear region of the fluid collection device is generally planar such that the dry adhesive region is generally planar.

17. A method of collecting fluid from a user, the method comprising:
positioning an opening of a fluid collection device at least proximate to a urethra of the user, the fluid collection device including a fluid impermeable barrier defining a chamber, the opening having fluid communication with the chamber, and an aperture having a conduit extending therethrough in fluid communication with the chamber;
positioning a garment on the user over the fluid collection device interfacing a dry adhesive region positioned on a rear region of the fluid impermeable barrier at least partially distal to the opening; and
collecting fluid voided or discharged by the user in the chamber of the fluid collection device.

18. The method of claim 17, further comprising securing the dry adhesive region to the rear region of the fluid impermeable barrier.

19. The method of claim 17, wherein the dry adhesive region includes one or more of: a biomimetic dry adhesive region; micro-scaled patterned wedges or nanometer-scaled wedges; hierarchical polymer micro-scaled hairs or nanometer-scaled hairs; wall-shaped hierarchical micro-scaled structures or nanometer-scaled structures; one or more micro-scaled fibrillar structures or nanometer-scaled fibrillar structures; micro-scaled brushes or nanometer-scaled brushes; micro-scaled mushroom shaped structures or nanometer-scaled mushroom shaped structures; micro-scaled patterned material or nanometer-scaled patterned material having polygonal or round patterns, round or polygonal dimples, and/or polygonal or round pillars; and/or micro or nanometer-scaled tubes.

20. The method of claim 17, wherein the fluid collection device includes a generally cylindrical shape such that the front region and the rear region of the fluid impermeable barrier are generally arched and the dry adhesive region arcs complementary to the rear region.

21. The method of claim 17, wherein the rear region of the fluid collection is generally planar such that the dry adhesive region is generally planar.

22. A method of forming a fluid collection device, the method comprising:
forming a fluid impermeable barrier having a rear region and a front region at least partially defining an opening and positioned on the fluid impermeable barrier to be at least proximate to a urethra of a user, the fluid impermeable barrier further at least partially defining a chamber and an aperture sized and dimensioned to receive a conduit therethrough;
positioning a fluid permeable body at least partially within the chamber to extend across at least a portion of the opening and configured to wick fluid away from the opening; and
positioning a dry adhesive region on the rear region of the fluid impermeable barrier at least partially distal to the opening to interface a garment worn by the user.

23. The method of claim 22, further comprising micro or nanopatterning geometric shapes with lithography or laser to form the dry adhesive region.

24. The method of claim 23, wherein positioning the dry adhesive region on the rear region of the fluid impermeable barrier includes securing a patch including the dry adhesive region on the rear region of the fluid impermeable barrier after micro or nanopatterning geometric shapes on the patch to form the dry adhesive region.

25. The method of claim 23, wherein micro or nanopatterning the geometric shapes to form the dry adhesive region includes micro or nanopatterning the geometric shapes directly on the rear region of the fluid impermeable barrier to form the dry adhesive region on the rear region of the fluid impermeable barrier.

26. The method of claim 22, wherein positioning a dry adhesive region on the rear region of the fluid impermeable barrier includes securing a patch including the dry adhesive region on the rear region of the fluid impermeable barrier.

27. The method of claim 22, wherein positioning a dry adhesive region on the rear region of the fluid impermeable barrier includes integrally forming the dry adhesive region directly on the rear region of the fluid impermeable barrier.

28. The method of claim 27, wherein the dry adhesive region includes one or more of: a biomimetic dry adhesive region; micro or nanometer-scaled wedges; hierarchical polymer micro or nanometer-scaled hairs; wall-shaped hierarchical micro or nanometer-scaled structures; one or more micro or nanometer-scaled fibrillar structures; micro or nanometer-scaled brushes; micro or nanometer-scaled mushroom shaped structures; micro or nanometer-scaled patterned material having polygonal or round patterns, round or polygonal dimples, and/or polygonal or round pillars; and/or micro or nanometer-scaled tubes.

29. The method of claim 22, wherein forming a fluid impermeable barrier includes forming the fluid impermeable barrier having a generally cylindrical shape such that the front region and the rear region of the fluid impermeable barrier are generally arched and the dry adhesive region arcs complementary to the rear region after positioning the dry adhesive region on the rear region of the fluid impermeable barrier.

30. The method claim 22, wherein forming a fluid impermeable barrier includes forming the fluid impermeable barrier with the rear region of being generally planar such that the dry adhesive region is generally planar after positioning the dry adhesive region on the rear region of the fluid impermeable barrier.
